(19) United States
(12) Patent Application Publication
(10) Pub. No.: US 2023/0193198 A1
(43) Pub. Date: Jun. 22, 2023

(54) METHOD FOR STABLY AMPLIFYING PLURIPOTENT STEM CELL

(71) Applicant: Gwo Xi Stem Cell Applied Technology Co., Ltd., Zhubei (TW)

(72) Inventors: Ming-Hsi Chuang, Zhubei (TW); Po-Cheng Lin, Zhubei (TW); Chia-Hsin Lee, Zhubei (TW); Mao-Hsuan Huang, Zhubei (TW)

(21) Appl. No.: 18/066,999

(22) Filed: Dec. 15, 2022

Related U.S. Application Data
(60) Provisional application No. 63/292,417, filed on Dec. 21, 2021.

(51) Int. Cl. C12N 5/074

(52) U.S. Cl. CPC C12N 5/0607; C12N 2533/74; C12N 2500/98; C12N 2500/12; C12N 2500/32; C12N 2523/00

(57) ABSTRACT

A method for stably amplifying a pluripotent stem cell comprises the following steps: (a) a cell implantation step: implanting pluripotent stem cells directly into a porous scaffold such that the porous scaffold contains 1×10⁴ or more of the pluripotent stem cells; and (b) a cell amplification step: immersing the porous scaffold in a specific culture medium which is xeno-free (XF) and performing amplification culture at an ambient temperature of 35.5-39.5° C. and a CO₂ concentration of 5% to obtain the amplified pluripotent stem cells, wherein the amplified pluripotent stem cells aggregate to present an embryoid body state. The amplification method of the present disclosure can easily obtain an excellent effect of increasing an amplification multiple of the pluripotent stem cells to about 3 times or more.

### CROSS-REFERENCE TO RELATED APPLICATION

The present application claims priority from U.S. Provisional Patent Application No. 63/292,417 filed on Dec. 21, 2021, the contents of which are incorporated herein by reference in their entirety.

### FIELD OF TECHNOLOGY

The present disclosure relates to an amplification culture method for a pluripotent stem cell, more particularly to an amplification culture method for a pluripotent stem cell by using a xeno-free (XF) culture medium. Cell death or failure of amplification culture caused by no use of an extracellular matrix (ECM), a feeder cell or a ROCK inhibitor and the like in a culture process is avoided.

### BACKGROUND

Pluripotent stem cells have a differentiation capacity second only to totipotent stem cells, include embryonic stem cells (ESCs) and induced pluripotent stem cells (iPSCs), which can provide an infinitely expanded source of cells and have a potential sufficient to differentiate into three embryonic layers and all tissues in an adult body. The pluripotent stem cells have extremely high application value in regenerative medicine, drug development, toxicity test, and gene research.

Since the pluripotent stem cells have the capacity of high differentiation, the pluripotent stem cells can be maintained in a non-differentiated state under a special culture medium, a culture environment and the like. For example, when the pluripotent stem cells are cultured without feeder cells or extracellular matrixes, a large amount or all of the pluripotent stem cells die and the culture fails. Therefore, in order to avoid such an undesirable situation, in the current technical field of cell amplification culture, the pluripotent stem cells are mostly subjected to amplification culture by using a two-dimensional culture system such as a feeder cell culture system, a culture system containing a ROCK inhibitor, or an extracellular matrix coating culture system.

The so-called “feeder cell culture system” is generally a two-dimensional culture system that utilizes, for example, mouse embryonic fibroblasts (MEFs) or human foreskin fibroblasts (HFFs) as feeder cells for growth culture of the pluripotent stem cells. Furthermore, the so-called “extracellular matrix coating culture system” is generally a two-dimensional culture system for culturing pluripotent stem cells in a culture medium to which a proper amount of essential growth factors have been added by using a culture tray having an extracellular matrix (ECM) coating. The so-called “culture system containing a ROCK inhibitor” is generally a two-dimensional culture system for growth culture of pluripotent stem cells in a culture medium supplemented with a ROCK inhibitor.

However, it is worth noting that since the feeder cell culture system, the culture system containing a ROCK inhibitor or the extracellular matrix coating culture system often uses xenogeneic animal materials, unnecessary safety issues must be faced, for example, including a wide variety of risks of large differences between each batch of produced cells, infection with pathogens brought by xenogeneic animals, or initiation of xenogeneic immune responses. Therefore, according to the Good Manufacturing Practice (GMP), the pluripotent stem cells cultured by using the feeder cell culture system, the culture system containing a ROCK inhibitor or the extracellular matrix coating culture system will not be used for clinical treatment purposes.

In the cell culture industry, it is expected to develop a method that can solve the problem that in the existing technology, under the condition of not using feeder cells or extracellular matrixes, a large number or all of the cells are easy to die during the culture and the culture fails. Besides, the method can stably amplify the pluripotent stem cells.

### SUMMARY

In view of the above, the present inventor focuses on the prior art and studies and develops a method for stably amplifying a pluripotent stem cell. The method comprises the following steps: filling a pluripotent stem cell into pores of a calcium alginate-based three-dimensional (3D) porous scaffold and performing a three-dimensional amplification culture of the pluripotent stem cell in a xeno-free cell culture environment in a culture medium free of animal serum or a xenogeneic animal material or any xenogeneic animal derived derivatives, specifically in a flat petri dish or a bioreactor. Besides, the pluripotent stem cell obtained after the three-dimensional amplification culture according to the method of the present disclosure can be directly used for differentiation culture for a purpose of cell therapy. So far, the present disclosure has been completed.

Therefore, the method for stably amplifying a pluripotent stem cell according to the present disclosure can avoid a problem of culture failure due to massive or total death of pluripotent stem cells in a culture environment without feeder cells or extracellular matrixes and can stably amplify pluripotent stem cells in large quantities. For example, according to the method for stably amplifying a pluripotent stem cell of the present disclosure, in a specific example, the amplification amount of pluripotent stem cells is approximately 3,000; and an amplification rate can reach about 30% per day. Therefore, the present disclosure can be very suitably used in various cell culture for a purpose of cell therapy.

Specifically, the present disclosure can provide an amplification culture method of a pluripotent stem cell. The method comprises the steps: (a) a cell implantation step: implanting a pluripotent stem cell directly into pores of a porous scaffold such that the porous scaffold contains 1×10⁴ or more of the pluripotent stem cells; and (b) a cell amplification step: immersing the porous scaffold in a specific culture medium which is xeno-free (XF) and performing amplification culture at an ambient temperature of 35.5-39.5° C. and a CO₂ concentration of 5% to obtain a large amount of the pluripotent stem cells, wherein the porous scaffold is mainly composed of calcium alginate and a porous particle with a micropore network structure. Further, the porous particle may be in the form of a solid, a non-liquid gel, a jelly, or a semi-solid; for example, a water gel. The specific culture medium is an E8 culture medium consisting of a DMEM/F12 culture medium, insulin, sodium selenite, transferrin, L-ascorbic acid, bFGF, TGF-β, and sodium bicarbonate (NaHCO₃).

According to one example of the present disclosure, the amplification amount of pluripotent stem cells is approximately 3,000; and an amplification rate can reach about 30% per day. Further, in one specific example, after culture for 7 days, the number of pluripotent stem cells is amplified to 2 times or more; after culture for 10 days, the number of pluripotent stem cells is amplified to about 3 times or more; and after culture for 14 days, the number of pluripotent stem cells is amplified to about 5 times or more.

According to one example of the present disclosure, the culture method does not need to use a feeder cell, an extracellular matrix coating or a ROCK inhibitor. Further, the feeder cell, extracellular matrix coating, or ROCK inhibitor can also be added as required without departing from the spirit of the present disclosure.

According to one example of the present disclosure, the porous scaffold is composed of calcium alginate.

According to one example of the present disclosure, a preparation method of the porous scaffold comprises the following steps: i, preparing a 1.0 wt %-5.0 wt % sodium alginate aqueous solution, injecting the solution into a 48-well culture plate with a volume of 1 mL/well, and then removing water by means of freeze-drying to form a spongy structure; and ii, immersing the spongy structure in a calcium chloride solution at a concentration of 1-2 wt % at a room temperature for crosslinking reaction, and after the reaction, performing gradient dehydration with different concentrations of ethanol to obtain the solid porous scaffold. The porous scaffold is a porous particle with a micropore network structure.

According to one example of the present disclosure, after step (b), the method further comprises (b-1) a cell release step: dissolving the porous scaffold with an ethylenediaminetetraacetic acid solution at a concentration of 50 mM, thereby releasing the pluripotent stem cells.

According to one example of the present disclosure, the specific culture medium is an E8 culture medium and at least contains a DMEM/F12 culture medium, insulin, selenium or selenide, transferrin, L-ascorbic acid, a basic fibroblast growth factor (bFGF, also known as fibroblast growth factor 2 (FGF2), and FGF-β), transforming growth factor beta (TGF-β), and sodium bicarbonate.

According to one example of the present disclosure, the amplified pluripotent stem cells aggregate to present an embryoid body appearance state.

According to one example of the present disclosure, the pluripotent stem cells are embryonic stem cells or induced pluripotent stem cells.

### BRIEF DESCRIPTION OF THE DRAWINGS

FIG. 1 shows morphology of pluripotent stem cells cultured in comparative example 1 and example 2, wherein (A) is comparative example 1 and (B) is example 2;

FIG. 2 shows morphology of pluripotent stem cells cultured in example 1 and example 2, wherein (A) is example 1 and (B) is example 2; and

FIG. 3 shows curve graphs of the number of cells cultured on the same days in examples 1 and 2.

### DESCRIPTION OF THE EMBODIMENTS

In order to enable the objective, technical features and advantages of the present disclosure to be more understood by a person skilled in the art to implement the present disclosure, the present disclosure is further illustrated by accompanying the appended drawings, specifically clarifying technical features and embodiments of the present disclosure, and enumerating better examples. In order to express the meaning related to the features of the present disclosure, the corresponding drawings herein below are not and do not need to be completely drawn according to the actual situation.

All technical and scientific terms used herein have the same meanings as those generally understood by a person of ordinary skill in the art to which the present disclosure pertains. Furthermore, as used herein, singular terms shall include a plural form and plural terms shall include a singular form, unless otherwise clearly contradicted by context.

Although the numerical ranges and parameters defining a broader scope of the present disclosure are all approximations. The related numerical values in the specific examples are presented herein as precisely as possible. However, any numerical value essentially inevitably contains standard deviations caused by an individual measuring method. As used herein, the term “about” generally refers to an actual value within plus or minus 10%, 5%, 1% or 0.5% of a specific value or range. Alternatively, the term “about” indicates that an actual value falls within an acceptable standard deviation of an average value, subjecting to consideration by a person with common knowledge in the art to which the present disclosure pertains. In addition to the examples or unless otherwise specified, all ranges, all the ranges, amounts, values and percentages (e.g., to describe amounts of materials, length of time, temperatures, operating conditions, quantitative proportion, and the like) herein used are to be understood as modified by the term “about”. Accordingly, unless indicated to the contrary, the numerical parameters set forth in the description and attached claims are approximations and may be changed as required. At least, these numerical parameters are to be understood as values obtained by the indicated number of significant digits and a general carry method.

In order to make the description of the present disclosure more detailed and complete, the following provides an illustrative description for the embodiments and specific examples of the present disclosure; but this is not the only form of implementing or using the specific examples of the present disclosure. The embodiments cover the features of the various specific examples as well as method steps and sequences for constructing and operating these specific examples. However, other examples may further be used to achieve the same or equivalent functions and step sequences.

First, the operation method of the present disclosure is described and comprises the following steps:

a cell implantation step S1: implanting a pluripotent stem cell directly into pores of a porous scaffold such that the porous scaffold contains the number of the pluripotent stem cells between 1×10⁴-1×10⁶. Further, it is worth noting that according to the technical idea of the present disclosure, the initial number of the pluripotent stem cells in the porous scaffold is not particularly limited, for example, the initial number of pluripotent stem cells may be greater than, equal to or less than 1×10⁴ according to practical needs; however, generally, the initial number of pluripotent stem cells is more than 1×10⁴ or more; preferably, the initial number of pluripotent stem cells is 5×10⁴ or more; more preferably, the initial number of pluripotent stem cells is 1×10⁵ or more; particularly preferably, the initial number of pluripotent stem cells is 5×10⁵ or more; and most preferably, the initial number of pluripotent stem cells is 1×10⁶ or more.

A cell amplification step S2: immersing the porous scaffold in a specific culture medium and performing amplification culture at an ambient temperature of 35.5-39.5° C. and a CO₂ concentration of 5% to obtain a large amount of the pluripotent stem cells.

In step S1, the porous scaffold is formed by crosslinking reaction of alginate and calcium chloride, and no extra extracellular matrix coating or feeder cell is required.

Alginic acid/alginate is a natural anionic polysaccharide that is mainly present in a cell wall or an intercellular space of brown algae. The sodium alginate is extracted and purified from brown algae, exists in the form of a sodium salt, and is a high molecular polymer consisting of two units of α-L-guluronic acid (G) and β-D-mannuronic acid (M) linked by a (1-4) chain. The unit arrangement of the sodium alginate varies according to the species, position and age of extracted seaweed, such as three blocks of MMM, GGG and MGM. Carboxyl (COO⁻) on the G unit of the sodium alginate is easy to generate a bonding effect with a divalent cation (such as Ca²⁺, Ba²⁺, Sr²⁺, and Pb²⁺), an intermolecular bridging effect (physical curing crosslinking) is generated, a hydrogel is formed, and an eggshell-box structure (egg-box junction) is generated between the carboxyl and the divalent cation. Therefore, a proportion of the G unit is relatively high and mechanical properties of the sodium alginate are also improved. However, when a proportion of the M unit is relatively high, the sodium alginate has a low viscosity and a weak mechanical strength. The physical properties, a gel-forming ability and strength of the sodium alginate depend on molecular weight, uronic acid composition, a proportion of three block arrangements (MMM, GGG and MGM), a source of a calcium ion, and a preparation method.

The United States Food and Drug Administration (FDA), as early as 1970, approved alginic acid generally recognized as safe (GRAS), i.e., a safe and non-toxic substance. The sodium alginate can be used for food, pharmaceuticals, drug use, and the like. Further, since a bonding action between alginic acid and a divalent cation causes a physical solidification and crosslinking. Therefore, in the present disclosure, after the porous scaffold mainly composed of calcium alginate is used for cell culture, when the porous scaffold is further washed with a divalent cation releasing compound, an excellent effect of quickly and easily removing the calcium alginate-based porous scaffold can be achieved. At the same time, the present disclosure can also be more advantageous for an application of a target cell culture for cell therapy and is further relatively safer. Further, the “divalent cation releasing compound” is not particularly limited. For example, a chelating agent is usually used as the divalent cation releasing compound in the present disclosure. For example, a chelating agent such as ethylenediaminetetraacetic acid (EDTA) or the like can be used.

The alginic acid can form a porous scaffold. The porous scaffold provides a three-dimensional environment that can allow cells to attach, proliferate, and differentiate. At the same time, a three-dimensional culture model is believed to better simulate an in-vivo cell growth environment and support expressions of specific genes and proteins. As mentioned above, for the alginic acid/sodium alginate with different physical properties, cell culture states are also different. The objectives of the present disclosure are that in a culture process, the porous scaffold can provide an environment for cell growth and after the culture is completed, the porous scaffold can be dissociated. Therefore, three sodium alginates with different properties were analyzed to evaluate which porous scaffold is suitable for the porous scaffold of the present disclosure:

Sodium alginate P1: high molecular weight (250-350 KDa), M/G proportion of 60-70/30-40, and viscosity of 200-450 mPa·s.

Sodium alginate P2: high molecular weight (90-180 KDa), M/G proportion of 60-70/30-40, and viscosity of 15-45 mPa·s.

Sodium alginate P3: high molecular weight (20-60 KDa), M/G proportion of 25-35/65-75, and viscosity of 5-7 mPa·s.

Among the three sodium alginates, sodium alginate P3 has the lowest viscosity and the lowest molecular weight, but a too-high dissociation speed, and therefore, before cell culture is completed, the cell scaffold is dissociated; sodium alginate P1 has the highest viscosity and the highest molecular weight, and provides a stable cell scaffold for cell growth, and then after the cell culture is completed, the cell scaffold is dissociated; and sodium alginate P2 can complete cell culture, and after the cell culture is completed, the porous scaffold can be dissociated. Sodium alginate P2 is used as a raw material of the porous scaffold in the examples of the present disclosure.

Further, in step S2, the specific culture medium is an E8 culture medium as shown in Table 1 and at least contains a DMEM/F12 culture medium, insulin, selenium or selenide, transferrin, L-ascorbic acid, a basic fibroblast growth factor (bFGF, also known as fibroblast growth factor 2 (FGF2), and FGF-β), transforming growth factor beta (TGF-β), and sodium bicarbonate.

It is worth noting that the specific culture medium (E8 culture medium) generally does not contain or is not supplemented with a ROCK inhibitor or a feeder cell in principle. However, it is also possible to further supplement or contain a ROCK inhibitor and/or a feeder cell as required. Further, according the present disclosure, the feeder cell may be, for example, a mouse embryonic fibroblast (MEF) or a human foreskin fibroblast (HFF).

In addition, in one example of the amplification culture method of a pluripotent stem cell of the present disclosure, for example, amplification culture in step (b) may be culture is firstly performed in the specific culture medium containing a ROCK inhibitor for a first period and then culture is performed in an E8 culture medium free of the ROCK inhibitor for a second period. Further, for example, the first period of the amplification culture in step (b) is at least 1 day; and the second period is at least 1 day.

In addition, it is worth noting that a surface of a used incubator (i.e. the porous scaffold) may also be treated. For example, in one example of the amplification culture method of a pluripotent stem cell of the present disclosure, between step (a) and step (b), the method may further comprise: (a-1) an extracellular matrix coating step: adding an extracellular matrix to the incubator to form an extracellular matrix coating. A human pluripotent stem cell (hPSC) has the ability to expand indefinitely and the potential to differentiate into all tissues in human body, Its surface markers represent SSEA4, Oct4, Sox2, and Nanog. Therefore, the human pluripotent stem cell is considered as important therapeutic strategy in regenerative medicine. In the past culture process, special factors must be added, such as a Rho kinase inhibitor (also known as a rho-associated protein kinase inhibitor or a ROCK inhibitor). Previous studies indicate that the ROCK inhibitor can maintain sternness of cells, is beneficial for viability and cell aggregation of hPSC, enhances the formation of an embryoid body (hPSC), improves a survival rate of human embryonic stem cells (hESCs) by preventing dissociation-induced apoptosis when the hESCs are dissociated into single cells, and thus increases cloning efficiency of the hESCs. Especially, the ROCK inhibitor is proved to be essential for maintaining viability of hESCs in an alginate microcapsule. However, the method of the present invention can amplify a pluripotent stem cell without the ROCK inhibitor.

Then, the present disclosure is described below with reference to the specific examples and comparative example.

### Comparative Example 1: Two-Dimensional Extracellular Matrix Coating Culture System

Human iPSCs were cultured using an extracellular matrix coating system. A coating of a petri dish was performed before the culture using an extracellular matrix solution (1% Geltrex), and a Geltrex solution was poured in the 60-mm petri dish in 1 ml each time, and the coating was performed at 37° C. for at least 12 h.

After the coating, the Geltrex solution was removed, induced pluripotent stem cells to be cultured by was added at 10⁴/well were added and immersed in an E8 culture medium (components were shown in Table 1) containing a ROCK inhibitor, and amplification culture was performed at 37° C. and under an environment of 5% CO₂.

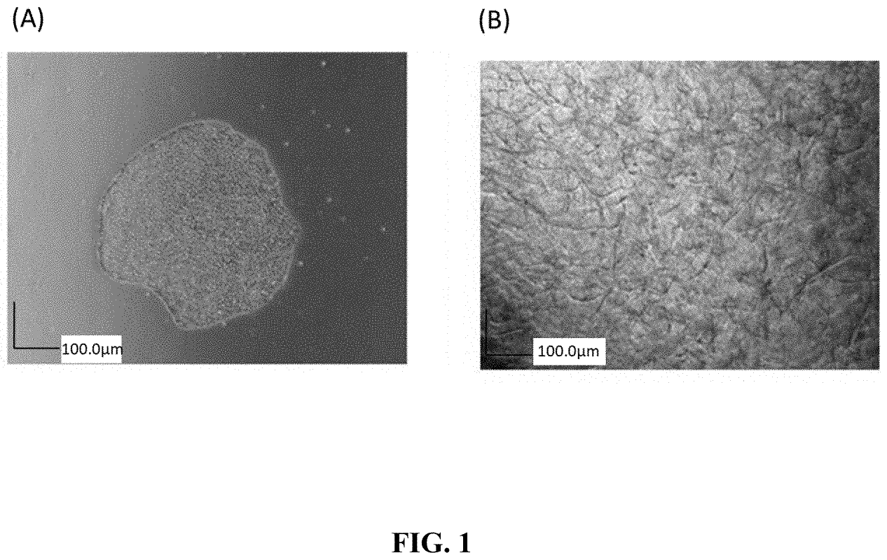

On the other day (day 2), a basic cell culture medium free of a ROCK inhibitor was used, the cells were cultured until day 14, and the amplified induced pluripotent stem cells were observed by a light microscopy (shown in view (A) in FIG. 1).

### Example 1: Three-Dimensional Porous Scaffold Culture System-Containing ROCK Inhibitor

Firstly, a porous scaffold was prepared. 1.5 wt % of a pharmaceutical-grade sodium alginate (Protanal® CR 8133, DuPont) powder was dissolved in deionized water and injected into a 48-well culture plate at a volume of 1 mL/well. The polymer solution was prepared into a porous scaffold by a freeze-drying technique. The porous scaffold is a porous particle with a micropore network structure. The porous scaffold was crosslinked in a 2% calcium chloride solution at a room temperature for 30-60 min, then sterilized with 75% ethanol, gradiently dehydrated with ethanol at different concentrations, irradiated with a 25-40 kGy Gamma ray, and stored at a room temperature for later use.

Then the porous scaffold was measured with a vernier caliper and the result was as follows: a diameter of 6.88±0.08 mm and a height of 6.43±0.09 mm. Further, it is worth noting that according to the technical idea of the present disclosure, an external dimension, a diameter, a height, etc. of a porous scaffold are not particularly limited, for example, a porous scaffold with a diameter of about greater than, equal to or less than 6.88 mm or more may be used according to practical needs; and a porous scaffold with a height of about greater than, equal to or less than about 6.43 mm may further be used.

Secondly, a pore size of pores of the porous scaffold was analyzed to be between 50 μm and 200 μm by a material specific surface area and pore size analyzer. Further, it is worth noting that according to the technical idea of the present disclosure, a pore size of pores of a porous scaffold, etc. are not particularly limited, for example, a porous scaffold having pores with a pore size of greater than, equal to or less than 50 μm, or about greater than, equal to or less than 200 μm may be used according to practical needs; however, generally, a porous scaffold having pores with a pore size of 25 μm or more and 200 μm or less is used; preferably, a porous scaffold having pores with a pore size of 35 μm or more to 185 μm or less is used; more preferably, a porous scaffold having pores with a pore size of 45 μm or more to 170 μm or less is used; particularly preferably, a porous scaffold having pores with a pore size of 55 μm or more and 155 μm or less is used; and most preferably, a porous scaffold having pores with a pore size of 65 μm or more and 1,140 μm or less is used.

Further, a porosity was analyzed to be 94.69±1.06% using a mercury porosimetry. Further, it is worth noting that according to the technical idea of the present disclosure, a porosity of a porous scaffold, etc. are not particularly limited, for example, a porous scaffold with a porosity of greater than, equal to or less than 90% may be used according to practical needs; however, generally, a porous scaffold with a porosity of 25% or more and 99% or less is used; preferably, a porous scaffold with a porosity of 30% or more and 95% or less is used; more preferably, a porous scaffold with a porosity of 35% or more and 90% or less is used; particularly preferably, a porous scaffold with a porosity of 40% or more and 85% or less is used; and most preferably, a porous scaffold with a porosity of 45% or more and 80% or less is used.

On the other hand, a porosity of a porous scaffold may also be measured by a liquid displacement method. Firstly, anhydrous ethanol with a volume of V0 was measured by a measuring cylinder, a sample of a porous scaffold was completely immersed, vacuumizing was performed for about 30 min, and at this time, a volume was recorded as V1; the sample of a porous scaffold was taken out, a volume of the remaining ethanol was recorded as V2, a porosity (P) of the sample of a porous scaffold was calculated by a formula of P=(V0−V2)/(V1−V2)×100%, the samples a total of 6 porous scaffolds were measured, and an average value was taken. According to the technical idea of the present disclosure, a porosity of a porous scaffold, etc. are not particularly limited, generally, a porous scaffold with a porosity of 35% or more is used; preferably, a porous scaffold with a porosity of 45% or more is used; more preferably, a porous scaffold with a porosity of 55% or more is used; particularly preferably, a porous scaffold with a porosity of 60% or more is used; and most preferably, a porous scaffold with a porosity of 65% or more is used.

Then the porous scaffold was placed in a 24-well culture tray, human iPSCs with the number of 1×10⁴/scaffold were planted in the porous scaffold, the porous scaffold was immersed in an E8 culture medium (components were shown in Table 1) containing a ROCK inhibitor, and amplification culture was performed at 37° C. and under an environment of 5% CO₂.

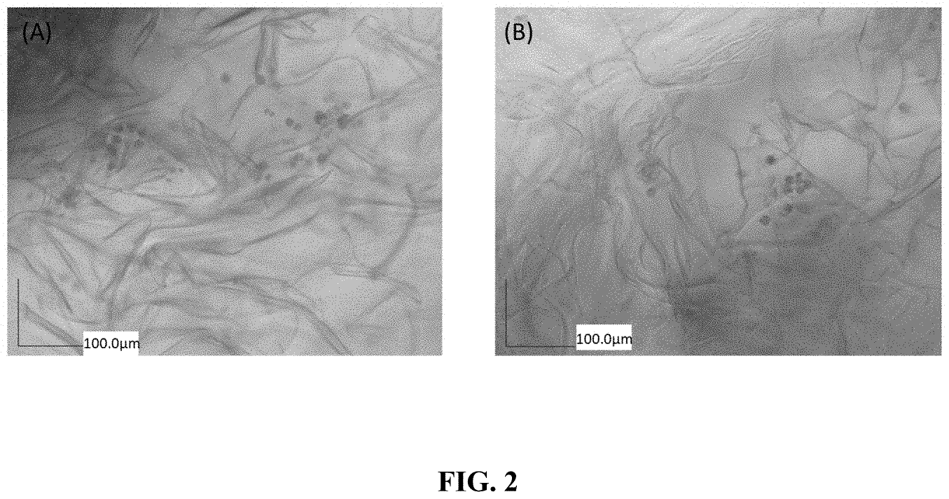

After the human iPSCs were cultured in the porous scaffold for different days, a 50 mM EDTA solution was used to dissolve the porous scaffold at 37° C. for 5 min, the human iPSCs were released from the porous scaffold, and the amplified induced pluripotent stem cells were observed by a light microscopy. A size of an embryoid body was calculated by a cell counter (Automated Brightfield Cell Counter Cellometer Auto T4) and the resulting embryoid body falls at 30-50 μm (shown in view (A) in FIG. 2).

### Example 2: Three-Dimensional Porous Scaffold Culture System-Free of a ROCK Inhibitor

Firstly, a porous scaffold was prepared. 1.5 wt % of a pharmaceutical-grade sodium alginate (Protanal® CR 8133, DuPont) powder was dissolved in deionized water and injected into a 48-well culture plate at a volume of 1 mL/well. The polymer solution was prepared into a porous scaffold by a freeze-drying technique. The porous scaffold is a porous particle with a micropore network structure. The porous scaffold was crosslinked in a 2% calcium chloride solution at a room temperature for 30-60 min, then sterilized with 75% ethanol, gradiently dehydrated ethanol at different concentrations, irradiated with a 25-40 kGy Gamma ray, and stored at a room temperature for later use.

Then the porous scaffold was placed in a 24-well culture tray, human iPSCs with the number of 1×10⁴/scaffold were planted in the porous scaffold, the porous scaffold was immersed in an E8 culture medium (components were shown in Table 1) free of a ROCK inhibitor, and amplification culture was performed at 37° C. and under an environment of 5% CO₂.

After the human iPSCs were cultured in the porous scaffold for different days, a 50 mM EDTA solution was used to dissolve the porous scaffold at 37° C. for 5 min, the human iPSCs were released from the porous scaffold, and the amplified induced pluripotent stem cells were observed by a light microscopy. A size of an embryoid body was calculated by a cell counter (Automated Brightfield Cell Counter Cellometer® Auto T4) and the resulting embryoid body falls at 30-50 μm (shown in view (B) in FIG. 2).

TABLE 1

| Component | Concentration | |
|---|---|---|
| DMEM/F12 culture medium | | |
| Insulin | 19.4 | mg/l |
| Sodium selenite | 14 | μg/l |
| Transferrin | 10.7 | mg/l |
| L-ascorbic acid magnesium | 64 | mg/l |
| bFGF | 100 | μg/l |
| TGF-β | 2 | μg/l |
| Sodium bicarbonate (NaHCO₃) | 543 | mg/l |

### Cell Morphology Analysis

FIG. 1 and FIG. 2 were referred. FIG. 1 showed morphology of pluripotent stem cells obtained by culture in comparative example 1 (show in view (A) in FIG. 1) and example 2 (shown in view (B) in FIG. 1) under a magnification of 40×.

It can be known by observing and comparing differences between view (A) in FIG. 1 and view (B) in FIG. 1 that in view (A) in FIG. 1 showed a dispersed state of pluripotent stem cells obtained by culture in comparative example 1; and in contrast, view (B) in FIG. 1 showed that the pluripotent stem cells obtained by culture in example 2 presented an aggregate embryoid body morphology, relatively more cell clusters existed, and the size of the embryoid body was uniform.

Since the induced pluripotent stem cells were generally in the form of embryoid bodies when subsequently subjected to differentiation culture, the induced pluripotent stem cells shown in view (A) in FIG. 1 cannot be directly used for differentiation culture, that is, the induced pluripotent stem cells cultured by the two-dimensional culture system of the prior art must be subjected to an aggregation procedure before used for differentiation culture. In contrast, the induced pluripotent stem cells shown in view (B) in FIG. 1 obtained by amplification culture according to example 2 of the present disclosure may be directly used for differentiation culture to obtain a specific organ tissue.

Secondly, FIG. 2 was referred. FIG. 2 showed morphology of pluripotent stem cells obtained by culture in example 1 (show in view (A) in FIG. 2) and example 2 (show in view (B) in FIG. 2) using the porous scaffold culture system under a magnification of 100×. It can be known by observing FIG. 2 that the pluripotent stem cells obtained by culture in example 1 presented an aggregate embryoid body morphology, relatively more cell clusters existed, and the size of the embryoid body was uniform. Therefore, similarly to example 2, the pluripotent stem cells obtained by culture in example 1 may also be directly used for differentiation culture to obtain a specific organ tissue.

### Cell Proliferation Analysis

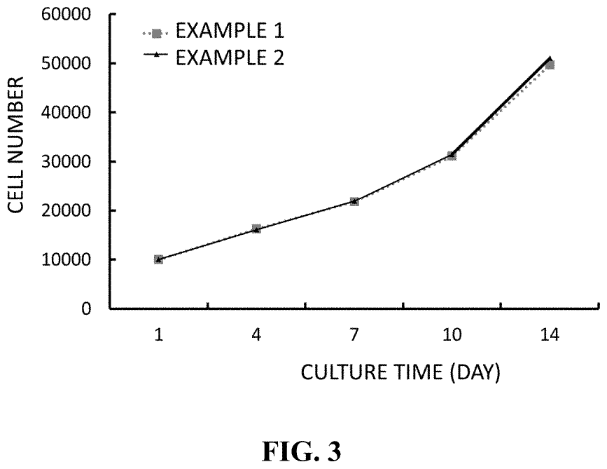

Then, the number of cells amplified in example 1 and example 2 was measured by trypan blue staining. Firstly, cells were stained with trypan blue by using a property that trypan blue permeates dead cells to develop color, but cannot permeate living cells to develop color since cell membranes of the living cells are intact. Then the number of cells was counted by an automatic particle counter (Coulter counter) to calculate the number of cells cultured for 1, 4, 7, 10, and 14 days in example 1 and example 2, respectively, and the result was recorded in Table 2. Further, the result was plotted into a cell number amplification curve shown in FIG. 3.

TABLE 2

Number of cells

| | Example 1 | Example 2 |
|---|---|---|
| | (containing a | (free of a |
| Culture days | ROCK inhibitor) | ROCK inhibitor) |
| 1 | 10,000 | 10,000 |
| 4 | 16,220 | 16,101 |
| 7 | 21,789 | 21,895 |
| 10 | 31,138 | 31,477 |
| 14 | 49,660 | 51,000 |
| Average cell | About 29.82 | About 30.85 |
| amplification | | |
| rate (%/day) | | |
| Average cell | About 2,982 | About 3,085 |
| amplification | | |
| amount (cell/day) | | |

As shown in Table 2 above, it can be seen that in example 1, after induced pluripotent stem cells were subjected to amplification culture for 4 days using a xeno-free culture medium containing a ROCK inhibitor and a three-dimensional porous scaffold, the number of the pluripotent stem cells was amplified to 16,220 and an amplification rate was 1.6 times or more; after culture for 7 days, the number of the pluripotent stem cells was amplified to 21,789 and an amplification rate was 2.17 times or more; after culture for 10 days, the number of the pluripotent stem cells was amplified to 31,138 and an amplification rate was 3.11 times or more; and after culture for 14 days, the number of the pluripotent stem cells was amplified to 49,660 and an amplification rate was 4.96 times or more. Further, the average cell amplification rate was about 29.82%/day; and the average cell amplification amount was about 2,982 cells/day.

Further, it may be further known that in example 2, after induced pluripotent stem cells were subjected to amplification culture for 4 days using a xeno-free culture medium and a three-dimensional porous scaffold, the number of the pluripotent stem cells was amplified to 16,101 and an amplification rate was 1.6 times or more; after culture for 7 days, the number of the pluripotent stem cells was amplified to 21,895 and an amplification rate was 2.18 times or more; after culture for 10 days, the number of the pluripotent stem cells was amplified to 31,477 and an amplification rate was 3.14 times or more; and after culture for 14 days, the number of the pluripotent stem cells was amplified to 51,000 and an amplification rate was 5.1 times or more. Further, the average cell amplification rate was about 30.85%/day; and the average cell amplification amount was about 3,085 cells/day.

Therefore, it can be known that in a traditional two-dimensional culture method free of an extracellular matrix coating, an extracellular matrix must be additionally coated, and the pluripotent stem cells may be amplified. However, the present disclosure utilizes a porous scaffold culture system to directly amplify pluripotent stem cells free of an extracellular matrix coating, indicating that the culture method of the present disclosure does not need to use any xenogeneic animal derived derivatives in a culture process. In contrast, the culture method of the present disclosure only requires a one-stage culture of a culture medium, which can rapidly amplify the induced pluripotent stem cells and can produce a large amount of pluripotent stem cells. Therefore, the culture method can simplify the culture process and make an application of a target cell culture for cell therapy to be relatively simpler and safer.

Further, FIG. 3 showed the cell number amplification curves after the amplification culture of the pluripotent stem cells in examples 1 and 2. A relationship between the number of cells after the amplification culture and the number of days of the amplification culture was analyzed by a quadratic regression analysis. The obtained relationship conformed to the following exponential relational expression (1):

y=6946.9e^{0.3893x}

R²=0.9939

In the formula, y represented the number of pluripotent stem cells after amplification culture; and x represented the number of days of the amplification culture.

Further, since R² in the exponential relational expression (1) was as high as 0.9939, the relationship between the number of days (x) of amplification culture and the number (y) of pluripotent stem cells after the amplification culture extremely highly conformed to the exponential relational expression (1). In conclusion, the content of the present disclosure has been exemplified with reference to the above examples, but the present disclosure is not limited to the embodiments. A person with common knowledge in the art may make various changes and modifications without departing from the spirit and scope of the present disclosure, for example, the technical contents exemplified in the above examples are combined or changed to new embodiments, and these embodiments are also regarded as one of the contents of the present disclosure. Accordingly, the scope to be protected in this case also includes the scope of aftermentioned claims and the scope defined therein.

### REFERENCE NUMERALS

None

1. A method for stably amplifying a pluripotent stem cell, wherein comprising at least the following steps:
(a) a cell implantation step: implanting pluripotent stem cells directly into pores of a porous scaffold such that the porous scaffold at least contains 1×10⁴ or more of the pluripotent stem cells; and
(b) a cell amplification step: immersing the porous scaffold obtained from step (a) that has been implanted with the pluripotent stem cells in a specific culture medium which is xeno-free (XF) and performing cell amplification culture at an ambient temperature of 35.5-39.5° C. and a CO₂ concentration of 5% to obtain the pluripotent stem cells amplified to a certain multiple or more, wherein
the porous scaffold is a porous particle with a micropore network structure and mainly composed of calcium alginate; and
the specific culture medium is an E8 culture medium consisting of a DMEM/F12 culture medium, insulin, sodium selenite, transferrin, L-ascorbic acid, bFGF, TGF-β, and sodium bicarbonate (NaHCO₃).

2. The amplification culture method of a pluripotent stem cell according to claim 1, wherein the porous scaffold is prepared by removing water from a sodium alginate aqueous solution by means of freeze-drying, and performing cross-linking with calcium chloride and dehydrating with ethanol.

3. The amplification culture method of a pluripotent stem cell according to claim 1, wherein the porous scaffold has a porosity of at least 65% or more, as measured by a liquid displacement method.

4. The amplification culture method of a pluripotent stem cell according to claim 1, wherein the pluripotent stem cells are embryonic stem cells or induced pluripotent stem cells.

5. The amplification culture method of a pluripotent stem cell according to claim 1, wherein the amplified pluripotent stem cells aggregate to present an embryoid body appearance state.

6. The amplification culture method of a pluripotent stem cell according to claim 1, after step (b), further comprising (b-1) a cell release step: dissolving the porous scaffold with a chelating agent at a concentration of 20-60 mM, thereby releasing the pluripotent stem cells.

7. The amplification culture method of a pluripotent stem cell according to claim 1, between step (a) and step (b), further comprising: (a-1) an extracellular matrix coating step: adding an extracellular matrix to the porous scaffold to form an extracellular matrix coating.

8. The amplification culture method of a pluripotent stem cell according to claim 1, wherein in step (b), a feeder cell is further added into the specific culture medium, and the feeder cell is a mouse embryonic fibroblast (MEF) or a human foreskin fibroblast (HFF).

9. The amplification culture method of a pluripotent stem cell according to claim 1, in step (b), further comprising adding a ROCK inhibitor into the specific culture medium; or after adding the ROCK inhibitor into the specific culture medium in advance and culturing the cells for a period of time, further removing the ROCK inhibitor.
